SEQUENCE LISTING (5 sequences)

SEQ ID NO: 1 - protein, 4 aa, Artificial Sequence
  opioid peptide
YGGF

SEQ ID NO: 2 - protein, 5 aa, Artificial Sequence
  opioid peptide
YGGFL

SEQ ID NO: 3 - protein, 4 aa, Artificial Sequence
  opioid peptide (endomorphin-2)
YPFF

SEQ ID NO: 4 - protein, 4 aa, Artificial Sequence
  opioid peptide (endomorphin-1)
YPWF

SEQ ID NO: 5 - protein, 5 aa, Artificial Sequence
  opioid peptide
YGGFM

(19) United States
(12) Patent Application Publication
(10) Pub. No.: US 2013/0072438 A1
(43) Pub. Date: Mar. 21, 2013

(54) OPIOID PEPTIDE ESTERS AND METHODS OF USE

(71) Applicant: Joel S. Goldberg, Hillsborough, NC (US)

(72) Inventor: Joel S. Goldberg, Hillsborough, NC (US)

(73) Assignee: The United States Government as represented by the Department of Veterans Affairs

(21) Appl. No.: 13/613,736

(22) Filed: Sep. 13, 2012

Related U.S. Application Data
(60) Provisional application No. 61/536,882, filed on Sep. 20, 2011.

(30) Foreign Application Priority Data
Jul. 24, 2012 (AU) 2012206979
Jul. 24, 2012 (CA) 2,783,359

(51) Int. Cl. A61K 38/05; A61P 25/04; A61K 38/14; C07K 5/065; C07K 9/00

(52) U.S. Cl. 514/18.4; 560/39; 548/533; 536/53; 514/21.91; 514/20.9; 552/544; 540/113

(57) ABSTRACT

Disclosed herein are opioid peptide conjugates (for example, opioid peptide esters). In some embodiments, the disclosed conjugates include an opioid peptide consisting of two to six amino acids and a moiety conjugated to the opioid peptide by an ester bond. In some examples, the moiety is an alcohol, a sugar, a lipid, or dehydroascorbic acid. Also disclosed are methods of altering nociception including administering an effective amount of one or more disclosed opioid peptide conjugates to a subject (such as a human subject).

### CROSS REFERENCE TO RELATED APPLICATIONS

This claims the benefit of U.S. Provisional Application No. 61/536,882, filed Sep. 20, 2011; Canadian Application No. 2,783,359, filed Jul. 24, 2012; and Australian Application No. 2012206979, filed Jul. 24, 2012, each of which is incorporated herein by reference in its entirety.

### FIELD

This disclosure relates to opioid peptides, particularly to opioid peptide esters and their use in altering nociception.

### BACKGROUND

For centuries, opioids derived from the poppy plant have been the mainstay of therapy for acute pain. In the last few decades, opioid analgesics have become the primary medications for the treatment of intractable chronic pain. However, in some patients the use of chronic opioid therapy has brought on a new set of problems. These include tolerance, addiction, pseudo-addiction, opioid induced hyperalgesia, bowel dysfunction, suppression of testosterone, cognitive impairment, substance abuse and diversion (Furlan et al., CMAJ 174:1589-1594, 2006). The most serious complication of chronic opioid therapy is the rising incidence of death from respiratory depression as a consequence of drug overdose of prescribed opioids (Shah et al., Addiction 103:126-136, 2008; Coolen, MMWR Morb. Mortal. Wkly. Rep. 58:1171-1175, 2009). Some medication-specific problems include prolongation of the QT interval from methadone (Pearson and Woosley, Pharmacoepidemiol. Drug Saf 14:747-753, 2005; Krantz et al.,

Pharmacotherapy 23:802-805, 2003; Huh et al., Korean J. Anesthesiol. 58:338-343, 2010), seizures from normeperidine, a metabolite of meperidine (Hagmeyer et al., Ann. Pharmacother. 27:29-32, 1993; Marinella, South. Med. J. 90:556-558, 1997), convulsions from morphine-3 glucoronide, a metabolite of morphine (Smith, Clin. Exp. Pharmacol. Physiol. 27:524-528, 2000), and fluctuating bioavailability from use of transdermal fentanyl (Med. Lett. Drugs Ther. 51:64, 2009; Heiskanen et al., Pain 144:218-222, 2009; Jumbelic, Am. J. Forensic Med. Pathol. 31:18-21, 2010).

The development of synthetic opioids, such as fentanyl, meperidine, and methadone has improved the bioavailability, potency, and to some degree the side effects profile of analgesics. However, one of the major obstacles in the development of synthetic opioids has been developing a stable medication that can cross the blood-brain barrier (BBB) (Witt and Davis, AAPSJ 8:E76-88, 2006; Gentilucci, Curr. Top. Med. Chem. 4:19-38, 2004).

### SUMMARY

Disclosed herein are opioid peptide conjugates (for example, opioid peptide esters (OPEs)) which can be used to modify nociception (for example, to produce analgesia or hyperalgesia). In some embodiments, the disclosed conjugates include an opioid peptide consisting of two to six amino acids and a moiety conjugated to the opioid peptide by an ester bond. In some examples, the moiety is an alcohol, a sugar, a lipid, or dehydroascorbic acid. In some embodiments, the disclosed conjugates are capable of crossing the blood-brain barrier (BBB).

Also disclosed herein are methods of altering (for example, increasing or decreasing) nociception in a subject (such as a human subject). In some embodiments, the methods include administering an effective amount of a disclosed OPE to the subject. In some examples, the OPE is administered parenterally or orally.

The foregoing and other features of the disclosure will become more apparent from the following detailed description.

### Sequence Listing

The nucleic acid and amino acid sequences listed herein and included in the accompanying Sequence Listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

The Sequence Listing is submitted as an ASCII text file in the form of the file named Sequence_Listing.txt, which was created on Sep. 11, 2012, and is 1,199 bytes, which is incorporated by reference herein.

SEQ ID NOs: 1-5 are exemplary opioid peptide amino acid sequences.

### DETAILED DESCRIPTION

### I. Abbreviations

BBB blood-brain barrier

CNS central nervous system

Da Daltons

OPE opioid peptide ester

### II. Terms

Unless otherwise noted, technical terms are used according to conventional usage. Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Although methods and materials similar or equivalent to those described herein can be used in the practice or testing of the present disclosure, suitable methods and materials are described below.

All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Administering: To provide or give a subject an agent, such as a therapeutic agent, by any effective route. Exemplary routes of administration include, but are not limited to, injection (such as subcutaneous, intramuscular, intradermal, intraperitoneal, intrathecal, epidural, and intravenous), oral, intraductal, sublingual, rectal, transdermal, intranasal, vaginal and inhalation routes.

Analgesic agent: An analgesic agent may be either an anesthetic that provides insensitivity to pain, or an agent that diminishes sensitivity to pain without necessarily ablating pain perception entirely. In some examples, the disclosed OPEs are non-anesthetic analgesic agents. In other examples, the disclosed OPEs are anesthetic.

Blood-brain barrier (BBB): The barrier formed by epithelial cells in the capillaries that supply the brain and central nervous system. This barrier selectively allows entry of substances such as water, oxygen, carbon dioxide, and nonionic solutes such as glucose, alcohol, and general anesthetics, while blocking entry of other substances. Some small molecules, such as glucose and amino acids, are taken across the barrier by specific transport mechanisms.

Conjugate or Bio-conjugate: A compound having a molecule (for example, a biomolecule, such as an opioid peptide) effectively coupled to another molecule or moiety (for example, a small molecule such as an alcohol, sugar, or lipid), either directly or indirectly, by any suitable means. In some examples, the molecule can be directly covalently coupled to a nanoparticle (such as by an ester bond).

Conjugating, joining, bonding or linking: Coupling a first unit to a second unit. This includes, but is not limited to, covalently bonding one molecule to another molecule (for example, directly or via a linker molecule), noncovalently bonding one molecule to another (e.g. electrostatically bonding), non-covalently bonding one molecule to another molecule by hydrogen bonding, non-covalently bonding one molecule to another molecule by van der Waals forces, and any and all combinations of such couplings

Effective amount: An amount of a compound or a combination of compounds sufficient to achieve a desired effect, for example to treat or inhibit a disease or condition in a subject. The amount of a compound or combination of compounds which is an effective amount will vary depending on the compound and the desired effect. An effective amount can be determined by one of ordinary skill in the art.

Nociception: The neural processes of encoding and processing noxious stimuli, for example the afferent activity produced in the peripheral and central nervous system by stimuli that have the potential to damage tissue. This activity is initiated by nociceptors (also called pain receptors), that can detect mechanical, thermal or chemical changes above a set threshold. Once stimulated, a nociceptor transmits a signal along the spinal cord, to the brain. In some embodiments, nociception refers to the perception of pain.

Opioid Peptide: A short sequence of amino acids that binds to one or more opioid receptors. In some embodiments, opioid peptides are naturally occurring peptides, for example, endorphins, enkephalins, dynorphins, adrenorphin, amidorphin, casomorphin (from milk), gluten exorphin (from gluten), gliadorphin/gluteomorphin (from gluten), and rubiscolin (from spinach). In other embodiments, opioid peptides are synthetic or non-naturally occurring peptides, for example of two or more (such as two to six) amino acids in length, such as two to six amino acids of a naturally occurring opioid peptide.

Pharmaceutically acceptable carrier: The pharmaceutically acceptable carriers useful in this disclosure are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the agents disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Subject: Living multi-cellular vertebrate organisms, a category that includes both human and non-human mammals.

### III. Opioid Peptide Conjugates

Disclosed herein are opioid peptide conjugates, such as opioid peptide esters (OPEs). The conjugates are useful in methods for altering nociception in a subject, for example increasing or decreasing nociception, and in some examples can be analgesic agents. In some embodiments, the disclosed conjugates are capable of crossing the BBB. Also disclosed herein are methods for altering nociception in a subject (such as a human subject) including administering an effective amount of one or more OPEs to the subject. In some examples, the methods include decreasing nociception by the subject (for example, decreasing pain or pain perception). In other examples, the methods include increasing nociception by the subject (for example, increasing perception of or sensitivity to pain or a noxious stimulus).

In some embodiments, the disclosed conjugates include an opioid peptide conjugated to a moiety by an ester bond. In some examples, the opioid peptide includes or consists of two to six amino acids (such as 2, 3, 4, 5, 6, or more amino acids). In other examples, the opioid peptide is about 100 to 700 Daltons (such as 200 to 600 Da, 100 to 500 Da, 250 to 500 Da, 300 to 500 Da, 350 to 500 Da, 400 to 500 Da, 600 to 700 Da, 200 to 400 Da, or 200 to 300 Da). In some examples, the conjugate is hydrolyzed by esterases (for example in the cerebrospinal fluid), releasing the active opioid peptide.

In particular examples, the opioid peptide includes or consists of Tyr-Gly, Tyr-Pro, Tyr-Ala, Tyr-Gly-Gly, Tyr-Pro-Phe, Tyr-Pro-Trp, Tyr-Gly-Gly-Phe (SEQ ID NO: 1), Tyr-Gly-Gly-Phe-Leu (SEQ ID NO: 2), Tyr-Pro-Phe-Phe (SEQ ID NO: 3), Tyr-Pro-Trp-Phe (SEQ ID NO: 4) or Tyr-Gly-Gly-Phe-Met (SEQ ID NO: 5). In a particular example, the opioid peptide consists of Tyr-Gly or Tyr-Pro. In some examples, the opioid peptide includes one or more modifications, for example to increase lipophilicity of the peptide. In one example, the hydroxyl group of a tyrosine residue is acetylated in an opioid peptide. In other examples, the opioid peptide includes one or more modifications to increase stability of the peptide or OPE. In some embodiments, the opioid peptide includes one or more d-amino acids (such as 1, 2, 3, 4, 5, or 6 D-amino acids).

In some embodiments, the opioid peptide is conjugated to a moiety by an ester bond. In some examples, the moiety is an alcohol, a sugar, a lipid, or dehydroascorbic acid. One of ordinary skill in the art can select an appropriate moiety to include in the conjugate with an opioid peptide. In some examples, the moiety is one that can facilitate transport across or through the BBB, for example through a cellular transporter (for example, via a glucose transporter) or due to lipophilicity (for example, a lipid). In addition, in some examples the moiety is one that is familiar to the central nervous system (CNS), for example, the moiety is one known to be generally non-toxic to the CNS. In some embodiments, the moiety is an alcohol, such as ethanol, diethylaminoethanol, benzyl alcohol, propanol, or butanol. In one non-limiting example, the moiety is ethanol. In other embodiments, the moiety is a sugar, such as glucose or fructose. Appropriate sugars include those that can be transported across the BBB by one of the family of glucose transporters (e.g., GLUT1 to GLUT5). In some examples, the moiety is directly conjugated to an opioid peptide. In other examples, the moiety and the opioid peptide are conjugated via a linker molecule.

In some non-limiting embodiments, the OPE includes Tyr-Pro-Ethyl, Tyr-Gly-Ethyl, Tyr-Pro-3-Glucosyl, Tyr-Gly-3-Glucosyl, Tyr-Pro-Cholestryl, or Tyr-Gly-Cholestryl. In one particular example, the OPE comprises Tyr-Pro-Ethyl having the structure:

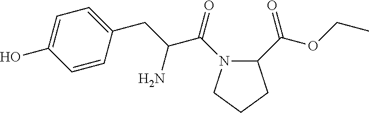 [SMILES: CCOC(=O)C1CCCN1C(=O)C(N)Cc1ccc(O)cc1]

The disclosed OPEs can be synthesized by methods known to one of ordinary skill in the art. Opioid peptides can be produced by standard techniques, such as solid phase synthesis (for example, utilizing an automated peptide synthesizer or manual peptide synthesis), standard solution synthesis or simultaneous multiple peptide synthesis. See, e.g., Merrifield, J. Am. Chem. Soc. 85:2149-2154, 1964; Bodanszky, Principles of Peptide Synthesis, 2^{nd} Edition, Springer, 1993; Pennington and Dunn, Peptide Synthesis Protocols, Humana Press, 2005. OPEs can be synthesized by any method of ester synthesis known in the art or discovered in the future. See, e.g., U.S. Pat. No. 5,051,448, incorporated herein by reference. As an example, an OPE can be prepared by simple esterification of an opioid peptide and a moiety (such as an alcohol) in the presence of a strong acid. In another example, an OPE can be prepared by converting an opioid peptide to the anhydride (for example utilizing a carbodiimide, such as dicyclohexylcarbodiimide) and subsequent esterification of the anhydride. In some examples, reactive groups of the opioid peptide can be protected prior to esterification. For example, if the opioid peptide includes an OH group that is not to be esterified, it can be protected, for example by forming an acetonide derivative. In addition, the amino group of an opioid peptide can be protected, for example, by forming of a t-butoxycarbonyl derivative. Following esterification, protecting groups can be removed by standard techniques, for example treatment with strong acid.

In some examples, the OPE is a conjugate that is a potentially orally active drug. One of ordinary skill in the art can identify conjugates that are potentially orally active. In some examples, the OPE satisfies Lipinski's Rule (Lipinski et al., Adv. Drug Del. Rev. 46:3-26, 2001). For example, the conjugate satisfies at least three of: 1) not more than five hydrogen bond donors, 2) not more than ten hydrogen bond acceptors, 3) a molecular mass of not more than 500 Da, and 4) an octanol-water partition coefficient log P not greater than five. In other examples, the conjugate satisfies an alternative set of criteria for potentially orally active compounds, such as 1) partition coefficient log P of −0.4 to 5.6, 2) molar refractivity from 40 to 130, 3) molecular weight from 160 to 500 Da, 4) 20 to 70 atoms, and 5) polar surface area no greater than 140 Å² (see, e.g., Ghose et al., J. Combin. Chem. 1:55-68, 1999). In further examples, a conjugate is identified as potentially capable of crossing the BBB, for example, satisfying at least three of: 1) octanol-water partition coefficient log P not greater than five, 2) molecular weight of not more than 400 Da, 3) not more than three hydrogen bond donors, and 4) not more than seven hydrogen bond acceptors. In silico prediction of whether a compound can potentially cross the BBB can also be utilized (e.g., Ekins and Tropsha, Pharm. Res. 26:1283, 2009; Goodwin and Clark, J. Pharmacol. Exp. Ther. 315:477-483, 2005; Doniger et al., J. Comp. Biol. 9:849-864, 2004; ACD/Labs ADME Suite (Toronto, Canada)). One of ordinary skill in the art can identify potentially orally active conjugates or conjugates potentially able to cross the BBB utilizing one or more of these sets of criteria.

The OPEs thus identified can serve as conventional “lead” compounds or can themselves be used as potential or actual therapeutics. One of ordinary skill in the art will appreciate that conjugates that do not strictly conform to these criteria may also be potentially active drugs and/or potentially able to cross the BBB and the disclosed conjugates are not strictly limited to those that meet these criteria.

### IV. Pharmaceutical Compositions and Methods of Use

Pharmaceutical compositions that include an opioid peptide conjugates, such as the OPEs disclosed herein can be formulated with an appropriate pharmaceutically acceptable carrier, depending upon the particular mode of administration chosen. The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually comprise injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate.

In some examples, the pharmaceutical composition including one or more OPEs includes injectable preparations such as sterile suspensions, solutions or emulsions of the active compound(s) in aqueous or oily vehicles. The compositions may also contain formulating agents, such as suspending, stabilizing and/or dispersing agents. The formulations for injection may be presented in unit dosage form, e.g., in ampules or in multidose containers, and may contain added preservatives. Alternatively, an injectable formulation may be provided in powder form for reconstitution with a suitable vehicle, including but not limited to sterile pyrogen free water, buffer, or dextrose solution before use. In such examples, the composition may be dried by any art-known technique, such as lyophilization, and reconstituted prior to use.

Pharmaceutical compositions including the disclosed OPEs for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavoring agents, coloring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatin or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monostearate or glyceryl distearate may be employed. Pharmaceutical compositions for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium such as peanut oil, liquid paraffin or olive oil. Suitable pharmaceutical compositions and unit dose forms may be prepared using conventional methods known to those in the field of pharmaceutical formulation and described in the pertinent texts and literature (See, e.g., Remington: The Science and Practice of Pharmacy, The University of the Sciences in Philadelphia, Editor, Lippincott, Williams, & Wilkins, Philadelphia, Pa., 21^{st} Edition, 2005).

Liquid preparations for oral administration include elixirs, solutions, syrups or suspensions, or a dry product for constitution with water or other suitable vehicle before use. Such liquid preparations may be prepared by conventional means with pharmaceutically acceptable additives such as suspending agents (such as sorbitol syrup, cellulose derivatives or hydrogenated edible fats); emulsifying agents (for example, lecithin or acacia); non-aqueous vehicles (such as almond oil, oily esters, ethyl alcohol, or fractionated vegetable oils); and preservatives (for example, methyl or propyl-p-hydroxybenzoates or sorbic acid). Liquid preparations may also contain buffer salts, preservatives, flavoring, coloring and sweetening agents as appropriate.

In some embodiments, the OPE is included in a controlled release formulation, for example, a microencapsulated formulation. Various types of biodegradable and biocompatible polymers can be used, and methods of encapsulating a variety of synthetic compounds, proteins and nucleic acids, have been well described in the art (see, for example, U.S. Pat. Publication Nos. 2007/0148074; 2007/0092575; and 2006/0246139; U.S. Pat. Nos. 4,522, 811; 5,753,234; and 7,081,489; PCT Publication No. WO/2006/052285; Benita, Microencapsulation: Methods and Industrial Applications, 2^{nd} ed., CRC Press, 2006).

In other embodiments, the OPE is included in a nanodispersion system. Nanodispersion systems and methods for producing such nanodispersions are well known to one of ordinary skill in the art. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In some examples, the nanodispersion is prepared using the solvent evaporation method. See, e.g., Kanaze et al., Drug Dev. Indus. Pharm. 36:292-301, 2010; Kanaze et al., J. Appl. Polymer Sci. 102:460-471, 2006.

In other examples, the disclosed compounds and pharmaceutical compositions are formulated as a depot preparation for administration by implantation or intramuscular injection. The active ingredient may be formulated with suitable polymeric or hydrophobic materials (for example, as an emulsion in an acceptable oil) or ion exchange resins, or as sparingly soluble derivatives, (such as a sparingly soluble salt). Alternatively, transdermal delivery systems manufactured as an adhesive disc or patch which slowly releases the active compounds for percutaneous absorption are used. Permeation enhancers may be used to facilitate transdermal penetration of the composition. Transdermal patches are described for example, in U.S. Pat. No. 5,407,713.; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

In some examples, an OPE conjugate includes a pharmaceutically acceptable salt of such compounds. “Pharmaceutically acceptable salts” of the presently disclosed compounds include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts are also inclusive of the free acid, base, and zwitterionic forms. Description of suitable pharmaceutically acceptable salts can be found in Handbook of Pharmaceutical Salts, Properties, Selection and Use, Wiley VCH (2002).

The dosage form of the pharmaceutical compositions will be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate. Actual methods of preparing such dosage forms are known, or will be apparent, to those skilled in the art.

The compounds of this disclosure can be administered to humans or other animals on whose tissues they are effective in various manners such as orally, intravenously, intramuscularly, intraperitoneally, intranasally, intradermally, transdermally, intrathecally, epidurally, sublingually, subcutaneously, via inhalation or via suppository. In one non-limiting example, the compound is administered orally. In another non-limiting example, the compound is administered intravenously, transdermally, intrathecally, epidurally, or sublingually. The particular mode of administration and the dosage regimen is selected by the attending clinician, taking into account the particulars of the case (e.g. the subject, the disease or condition involved, and whether the treatment is prophylactic). Treatment can involve daily or multi-daily doses of compound(s) over a period of a few days to months, or even years. One of ordinary skill in the art can identify appropriate doses for the OPEs of use in the disclosed methods. The amount administered will be dependent on factors such as the subject being treated, the type and severity of the condition, and the mode of administration.

A pharmaceutical composition that includes one or more OPEs can be formulated in unit dosage form, suitable for individual administration of precise dosages. In one specific, non-limiting example, a unit dosage contains from about 10 μg to about 5 g or more of one or more OPEs (such as about 50 μg to about 1 mg, about 100 μg to about 10 mg, about 1 mg to about 2.5 g, about 10 mg to about 1 g, or about 100 mg to about 500 mg). In some examples, a unit dosage contains about 10 μg or more of one or more OPEs (such as about 10 μg, 25 μg, 50 μg, 75 μg, 100 μg, 200 μg, 250 μg, 500 μg, 750 μg, 1 mg, 2.5 mg, 5 mg, 7.5 mg, 10 mg, 25 mg, 50 mg, 100 mg, 250 mg, 500 mg, 750 mg, 1 g, 1.5 g, 2 g, 2.5 g, 3 g, 4 g, 5 g, or more). The amount of active compound administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

In some embodiments, an effective amount of one or more disclosed OPE is administered to a subject, thereby altering nociception in the subject. In some examples, administration of an OPE to a subject decreases nociception or pain perception (for example, the OPE is an analgesic agent). In other examples, administration of an OPE to a subject increases nociception or pain perception (for example, the OPE is a hyperalgesic agent). In some examples, an effective amount of an OPE is about 1 μg/kg to about 100 mg/kg (for example, aboutl μg/kg to about 10 mg/kg, about 10 μg/kg to about 5 mg/kg, about 100 μg/kg to about 1 mg/kg, about 1 mg/kg to about 50 mg/kg, about 10 mg/kg to about 25 mg/kg, or about 20 mg/kg to about 100 mg/kg). In some examples, an effective amount is about 1 μg/kg or more of an OPE (such as about 1 μg/kg, 5 μg/kg, 10 μg/kg, 25 μg/kg, 50 μg/kg, 75 μg/kg, 100 μg/kg, 250 μg/kg, 500 μg/kg, 750 μg/kg, 1 mg/kg, 2 mg/kg, 3 mg/kg, 4 mg/kg, 5 mg/kg, 6 mg/kg, 7mg/kg, 8 mg/kg, 9 mg/kg, 10 mg/kg, 15 mg/kg, 20 mg/kg, 50 mg/kg, or more). In a specific example, an effective amount of an OPE is about 5 mg/kg to about 20 mg/kg, such as about 10 mg/kg. In another specific example, an effective amount of an OPE is about 1 μg/kg to about 1 mg/kg, such as about 10 μg/kg to 100 μg/kg or about 200 μg/kg to 600 μg/kg. One of ordinary skill in the art can extrapolate from an animal dose (such as a rat or mouse) to an appropriate human dose, such as for use in clinical trials for determining pharmacokinetics and dosing (see, e.g., Reagan-Shaw et al., FASEB J. 22:659-661, 2008).

An effective amount of an OPE can be the amount of OPE necessary to alter nociception (such as to treat or inhibit pain, for example to decrease pain or pain perception) in a subject. An effective amount of an OPE can be administered in a single dose, or in several doses, for example weekly, bi-weekly, daily, or 2, 3, 4, or 5 more times daily, during a course of treatment. One of ordinary skill in the art can determine the effective amount of an OPE based for example, on the subject being treated, the severity and type of the affliction, the manner of administration, and the physicochemical properties of the OPE.

In particular examples, prior to, during, or following administration of an effective amount of an OPE, the subject can receive one or more other therapies. In one example, the subject receives one or more additional treatments to alter nociception, such as one or more pain-relieving therapeutics other than an OPE (for example, a non-steroidal anti-inflammatory therapeutic). The combined administration of the OPE and additional pharmaceutical agents includes administering the additional agent either sequentially with the OPE, e.g., the treatment with one agent first and then the second agent, or administering both agents at substantially the same time, e.g., an overlap in performing the administration. With sequential administration a subject is exposed to the agents at different times so long as some amount of the first agent remains in the subject (or has a therapeutic effect) when the other agent is administered. The treatment with both agents at the same time can be in the same dose, e.g., physically mixed, or in separate doses administered at the same time.

The following examples are provided to illustrate certain particular features and/or embodiments. These examples should not be construed to limit the disclosure to the particular features or embodiments described.

### EXAMPLES

### Example 1

### Candidate Opioid Peptide Esters

Exemplary candidate low molecular weight opioid peptides are listed in ascending order (Table 1). Potential hydrogen bond donors (N+O with one or more H) and potential hydrogen bond acceptors (N+O) for each potential opioid peptide are listed as well as physical and chemical properties of the possible ester conjugates, glucose and cholesterol. Exemplary candidate OPE compounds are listed (Table 2).

TABLE 1
Molecular weights and potential hydrogen bonding of
constituents of possible OPE components

| | | H bond | |
|---|---|---|---|
| | | donor | H bond |
| | M.W. | (N + O)H | acceptor (N + O) |
| Tyr-Gly | 238 | 4 | 5 |
| Tyr-Pro | 278 | 2 | 4 |
| Tyr-Gly-Gly | 295 | 5 | 6 |
| Tyr-Gly-Gly-Phe (SEQ ID NO: 1) | 442 | 6 | 7 |
| Tyr-Gly-Gly-Phe-Leu (SEQ ID NO: 2) | 556 | 7 | 8 |
| Tyr-Pro-Phe-Phe-NH2 (endomorphin-2; | 572 | 5 | 6 |
| SEQ ID NO: 3) | | | |
| Tyr-Pro-Trp-Phe-NH2 (endomorphin-1; | 611 | 6 | 6 |
| SEQ ID NO: 4) | | | |
| Ethanol | 46 | 1 | 1 |
| Cholesterol | 387 | 1 | 1 |
| 3-Glucose | 180 | 5 | 6 |

TABLE 2
Candidate OPE compounds

| | | H bond donor* | H bond acceptor* | |
|---|---|---|---|---|
| | M.W. | (N + O)H | (N + O) | pKa |
| Tyr-Pro-Ethyl | 306 | 3 | 5 | 9.1 |
| Tyr-Gly-Ethyl | 266 | 1 | 4 | 9.1 |
| Tyr-Pro-3-Glucosyl | 440 | 6 | 9 | 9.1, 12.8 |
| Tyr-Pro-Cholestryl | 647 | 2 | 4 | 9.1 |
| Tyr-Gly-3-Glucosyl | 432 | 8 | 10 | 9.1, 12.8 |
| Tyr-Gly-Cholestryl | 607 | 4 | 5 | 9.1 |

*Assumes the —OH in Tyr is not acetylated

### Example 2

### In Vivo Testing of Candidate OPE

Three male Wistar rats at 8 weeks of age (Taconic Farms, Germantown, N.Y.) were utilized in the experiment. Each rat was grasped firmly, while the distal half of its tail was immersed in a liquid bath at a temperature of −20° C. Latency periods (in seconds) were measured from the time that tails were immersed to the time that the rat removed its tail from the liquid. Prior to dosing, rats were given three trials, approximately ten minutes apart. On the following day, rats were given intraperitoneal doses (0.5 mg, 1 mg, 2 mg) of Tyr-Pro-ethyl ester (Genscript, Piscataway, N.J.) and, after thirty minutes, another three trials were conducted (Tables 3-6). Post-dose trials were also approximately ten minutes apart. Animals were examined for acute adverse effects to both the cold liquid bath and the test article.

TABLE 3
Pre-treatment cold immersion response

| | Trial 1 | Trial 2 | Trial 3 | | |
|---|---|---|---|---|---|
| Animal | Latency (s) | Latency (s) | Latency (s) | Mean | SD |
| 1 | 17.8 | 18 | 16.8 | 17.5 | 0.6 |
| 2 | 7.4 | 5.6 | 8.4 | 7.1 | 1.4 |
| 3 | 14.2 | 9.8 | 9.7 | 11.2 | 2.6 |

TABLE 4
Pre-treatment means with longest latency period eliminated

| Animal | Mean | SD |
|---|---|---|
| 1 | 17.3 | 0.7 |
| 2 | 6.5 | 1.3 |
| 3 | 9.8 | 0.1 |

TABLE 5
Post-treatment cold immersion response

| | Dose | Trial 1 | Trial 2 | Trial 3 | | |
|---|---|---|---|---|---|---|
| Animal | (mg) | Latency (s) | Latency (s) | Latency (s) | Mean | SD |
| 1 | 1 | 13.2 | 22.1 | 11.5 | 15.6 | 5.693 |
| 2 | 2 | 4.7 | 4.7 | 12.2 | 7.2 | 4.3301 |
| 3 | 0.5 | 6.45 | 6.2 | 9.0 | 7.2167 | 1.5495 |

TABLE 6
Post-treatment means with longest latency period eliminated

| Animal | Mean | SD |
|---|---|---|
| 1 | 12.4 | 1.2 |
| 2 | 4.7 | 0.0 |
| 3 | 6.3 | 2.0 |

The data suggests that, at the doses given, Tyr-Pro-ethyl ester was able to cross the BBB, was hydrolyzed by esterases in the cerebrospinal fluid, and had some neurological effects on rats. This OPE may, in fact, increase sensitivity to noxious stimulus.

### Example 3

### Assessment of Analgesic Efficacy of OPEs

This example describes methods for the assessment of the efficacy of OPE administration for use as an analgesic agent.

Test subjects (such as laboratory mice or rats) are administered a dose of an OPE or vehicle at least 15 minutes (for example, 15, 30, 45, or 60 minutes) prior to testing. Doses of OPE include 0.1 mg/kg, 1 mg/kg, 2.5 mg/kg, 5 mg/kg, and 10 mg/kg.

In one example, a tail-flick test is performed (hot tail-flick test and/or cold tail-flick test), measuring the time taken by the subject to deflect the tail from warm water (50° C.) or cold water (−10° C.). An increase (such as a statistically significant increase) in the time until tail deflection (latency time) as compared to a control indicates that the OPE has an analgesic effect.

In a further example, a thermal sensitivity test is also used to assess analgesic effects of an OPE. A test subject is placed on a platform and a focused heat stimulus is delivered to one paw. The time until the paw is lifted is measured. An increase (such as a statistically significant increase) in the time until the paw is lifted (latency time) as compared to a control indicates that the OPE has an analgesic effect.

In another example, the formalin test is used to assess analgesic effects. Formalin (for example, 50 μl of 2.5% formalin) is injected in the intraplantar region of one paw. Animals are observed for pain-like behaviors, such as licking, biting or flinching. A decrease (such as a statistically significant decrease) in one or more of the pain-like behaviors as compared to a control indicates that the OPE has an analgesic effect.

In view of the many possible embodiments to which the principles of the disclosure may be applied, it should be recognized that the illustrated embodiments are only examples and should not be taken as limiting. Rather, the scope of the invention is defined by the following claims. We therefore claim as our invention all that comes within the scope and spirit of these claims.

We claim:

1. A conjugate comprising:
an opioid peptide consisting of two to six amino acids; and
a moiety conjugated to the opioid peptide by an ester bond, wherein the moiety comprises an alcohol, a sugar, a lipid, or dehydroascorbic acid.

2. The conjugate of claim 1, wherein the opioid peptide comprises:
(a) Tyr-Gly;
(b) Tyr-Pro;
(c) Tyr-Gly-Gly;
(d) Tyr-Pro-Phe
(e) Tyr-Pro-Trp
(SEQ ID NO: 1)
(f) Tyr-Gly-Gly-Phe;
(SEQ ID NO: 2)
(g) Tyr-Gly-Gly-Phe-Leu
(SEQ ID NO: 3)
(h) Tyr-Pro-Phe-Phe;
(SEQ ID NO: 4)
(i) Tyr-Pro-Trp-Phe;
or
(j) a combination of two or more thereof.

3. The conjugate of claim 1, wherein the alcohol comprises ethanol, diethylaminoethanol, benzyl alcohol, propanol, or butanol.

4. The conjugate of claim 1, wherein the sugar comprises glucose or fructose.

5. The conjugate of claim 1, wherein the lipid comprises cholesterol.

6. The conjugate of claim 1, wherein the conjugate comprises:
(a) Tyr-Pro-Ethyl;
(b) Tyr-Gly-Ethyl;
(c) Tyr-Pro-3-Glucosyl;
(d) Tyr-Gly-3-Glucosyl;
(e) Tyr-Pro-Cholestryl;
or
(f) Tyr-Gly-Cholestryl.

7. The conjugate of claim 1, wherein the conjugate has a molecular weight of about 100 to 700 Daltons.

8. The conjugate of claim 7, wherein the conjugate has a molecular weight of about 100-500 Daltons.

9. The conjugate of claim 1, wherein the conjugate is capable of crossing the blood-brain barrier.

10. A composition comprising the conjugate of claim 1 and a pharmaceutically acceptable carrier.

11. A method of altering nociception, comprising administering to a subject an effective amount of the conjugate of claim 1, thereby altering nociception in the subject.

12. The method of claim 11, wherein altering nociception comprises increasing nociception or decreasing nociception.

13. The method of claim 11, wherein the effective amount of the conjugate comprises about 1 μg/kg to 20 mg/kg.

14. The method of claim 13, wherein the effective amount of the conjugate comprises about 10 μg/kg to 1 mg/kg.

15. The method of claim 14, wherein the effective amount of the conjugate comprises about 0.2 mg/kg to 0.6 mg/kg.

16. The method of claim 11, wherein administering the conjugate comprises intravenous, intrathecal, intraperitoneal, subcutaneous, oral, transdermal, epidural, or sublingual administration.

17. The method of claim 11, wherein the subject is human.
